(19) United States
(12) Patent Application Publication
(10) Pub. No.: US 2005/0251127 A1
(43) Pub. Date: Nov. 10, 2005

(54) Miniature ultrasonic transducer with focusing lens for intracardiac and intracavity applications

(76) Inventors: Jared Brosch, Cicero, IN (US); Andreas Hadjicostis, Carmel, IN (US); Grant Adam Morris, North Salem, IN (US)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 11/103,885

(22) Filed: Apr. 12, 2005

Related U.S. Application Data
(63) Continuation-in-part of application No. 10/686,120, filed on Oct. 15, 2003.
(63) Continuation-in-part of application No. 10/686,119, filed on Oct. 15, 2003.
(63) Continuation-in-part of application No. 10/868,415, filed on Jun. 14, 2004.

(51) Int. Cl. A61B 18/04

(52) U.S. Cl. 606/27

(57) ABSTRACT

One embodiment of the present invention includes advancing a transducer device through a passageway of a patient's body to a target location inside the body. The transducer device includes a piezoelectric element and an ultrasonic lens. The ultrasonic lens includes an inner surface defining a passage extending along a reference axis. The piezoelectric element is received in this passage and is acoustically coupled to the inner surface of the lens. The ultrasonic lens includes an outer surface opposite the inner surface, the outer surface defines a shape with a concave profile. While positioned at the target location, the transducer device generates ultrasonic energy and ablates tissue along at least a portion of a circumference around the transducer device at the target location by focusing the ultrasonic energy with the lens.

### CROSS-REFERENCE TO RELATED APPLICATIONS

The present application is related to commonly owned U.S. patent application Ser. No. 10/686,120 filed on Oct. 15, 2003; Ser. No. 10/686,119 filed on Oct. 15, 2003; and Ser. No. 10/868,415 filed on Jun. 14, 2004.

### BACKGROUND

The present invention relates to transducer devices for ultrasound applications, and more particularly, but not exclusively, relates to the fabrication, use, and structure of medical devices including one or more piezoelectric elements to generate ultrasonic energy and a lens for focusing the ultrasonic energy.

Heart disease represents one of the most common debilitating diseases among the elderly, and is a common cause of death. The mammalian heart typically has four chambers: two ventricles for pumping the blood and two atria, each for collecting the blood from the vein leading to it and delivering that blood to the corresponding ventricle. The left ventricle pumps blood to the vast bulk of the mammalian body. As a result, problems with the left ventricle or with the mitral valve, which leads from the left atrium into the left ventricle, can cause serious health problems. When it appears that a patient has inadequate blood circulation in a portion of his or her body, the left ventricle and the mitral valve are often suspect. Specifically diagnosing a problem with these structures; however, is not always an easy proposition. In fact, unnecessary surgeries are sometimes performed due to the difficulty of forming a proper diagnosis.

More particularly, cardiac arrhythmia—especially atrial fibrillation—persists as a common and dangerous medical aliment associated with abnormal cardiac chamber wall tissue. In patients with cardiac arrhythmia, abnormal regions of cardiac tissue do not follow the synchronous beating cycle associated with normally conductive tissue in patients with sinus rhythm. Instead, the abnormal regions of tissue aberrantly conduct to adjacent tissues, which disrupts the cardiac cycle causing an asynchronous rhythm. Such abnormal conduction is known to occur at various regions of the heart.

Irregular cardiac function and corresponding hemodynamic abnormalities caused by atrial fibrillation in particular can result in stroke, heart failure, and other medical problems. In fact, atrial fibrillation is believed to be a significant cause of cerebral stroke, wherein the hemodynamic abnormality in the left atrium caused by the fibrillatory wall motion precipitate the formation of thrombus within the atrial chamber. A thromboembolism is ultimately dislodged into the left ventricle which thereafter pumps the embolism into the cerebral circulation resulting in a stroke. Accordingly, numerous procedures for treating atrial arrhythmias have been developed, including pharmacological, surgical, and catheter ablation procedures.

Currently available methods for focusing ultrasonic energy and ablating tissue in the heart and/or vessels are not efficacious. One method uses sets of balloons to direct ultrasonic energy. A drawback of this method is that it can be imprecise and inaccurate in its ability to direct and focus ultrasonic energy to a specific location. Other methods employing lenses to focus ultrasonic energy are often inadequate for effectively ablating tissue due to the shapes and sizes of the lenses.

Accordingly, there is an interest in techniques, devices, and systems for intracardiac and/or intravascular tissue ablation with focused ultrasonic energy and further contributions in this area of technology are needed.

### SUMMARY

One embodiment of the present invention is a unique ultrasound method and device. Other embodiments include unique methods, systems, devices, and apparatus for focusing ultrasound and/or ablating tissue. As used herein, “ultrasound” and “ultrasonic” refer to acoustic energy waveforms having a frequency of more than 20,000 Hertz (Hz) through one or more media at standard temperature and pressure.

A further embodiment of the present invention includes a method involving advancing a transducer device through a passageway of a patient's body to a target location inside the body, the transducer device including a piezoelectric element and an ultrasonic lens. The ultrasonic lens includes an inner surface defining a passage extending along a reference axis. The piezoelectric element is received in this passage and is acoustically coupled to the inner surface. The ultrasonic lens includes an outer surface opposite the inner surface, the outer surface defines a shape with a concave profile. While positioned at the target location, the transducer device generates ultrasonic energy and ablates tissue along at least a portion of a circumference about the transducer device at the target location by focusing the ultrasonic energy with the lens.

Still a further embodiment includes: advancing a device through a passageway inside a patient's body towards a target location, maintaining the device in a selected position in the passageway relative to the target location, and ablating tissue at the target location by focusing ultrasonic energy generated with the device. The device includes a piezoelectric element and an ultrasonic lens. The ultrasonic lens includes an inner surface defining a passage and an outer surface defining a shape with a concave profile. The piezoelectric element is received in the passage and is a acoustically coupled to the inner surface. Ultrasonic energy generated with the piezoelectric element is focused by the concave profile with a focal link determined in accordance with this profile. In one particular form, the profile is revolved about a reference axis extending through the passage.

Another embodiment of the present application includes: providing a piezoelectric element that is approximately symmetric about a centerline axis longitudinally extending along the piezoelectric element, providing an ultrasonic lens that includes an inner surface defining a passage and an outer surface defining a shape with a concave profile, and which is approximately symmetric about a reference axis extending through the passage, placing the piezoelectric element in the passage to acoustically couple with the inner surface to provide an ablation assembly, and structuring the element and lens to focus ultrasonic energy in accordance with the concave profile to ablate material corresponding to a ring about the ablation assembly.

Still another embodiment includes a probe with a distal end portion opposite a proximal end portion that includes cabling and is structured to advance through a passageway of a patient's body to a target location including cardiac tissue, an ablation assembly included with the probe at the distal end portion to be carried therewith to the target location, and a controller structured to selectively activate and deactivate the piezoelectric element of the ablation assembly. The assembly includes a piezoelectric element coupled to the cabling and an ultrasonic lens. The ultrasonic lens includes an inner surface defining a cavity and an outer surface shaped with a concave profile. The piezoelectric element is positioned in the cavity and acoustically coupled to the inner surface of the lens. The controller is coupled to the cabling at the proximal end portion of the probe and is structured for placement external to the patient's body while the ablation assembly is positioned at the target location. The assembly is responsive to the controller to generate ultrasonic energy with the piezoelectric element and is structured to focus this energy at a focal length determined in accordance with concave profile and ablate cardiac tissue with the ultrasonic energy when the piezoelectric element is activated and the ablation assembly is positioned at the target location.

One object of the present invention is to provide a unique ablation device for ultrasound applications.

Another object of the present invention is to provide a unique method, system, device, or apparatus for focusing ultrasonic energy and/or ablating tissue using ultrasonic energy.

Further forms, objects, features, aspects, benefits, advantages, and embodiments of the present invention shall become apparent from the detailed description and drawings provided herewith.

### BRIEF DESCRIIPTION OF THE DRAWING

FIG. 1 is a schematic view of a system utilizing ultrasound.

FIG. 2 is partial, schematic sectional view of an ablation device included in the system of FIG. 1.

FIG. 3 is partial, schematic sectional view of the ablation device of FIG. 2 taken along section line 3-3 shown in FIG. 2.

FIG. 4 is a partial, sectional, schematic view of a distal end portion shown in the system of FIG. 1 that includes the ablation device of FIG. 2.

FIG. 5 is a partial, sectional view of another ablation device that can be used as an alternative to the ablation device of FIG. 2 shown in FIG. 4.

FIG. 6 is a partial, schematic view of an ablation assembly to illustrate certain operational aspects.

### DETAILED DESCRIPTION OF SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

One embodiment of the present invention includes a method involving advancing a transducer device through a passageway of a patient's body to a target location inside the body, the transducer device including a piezoelectric element and an ultrasonic lens. The ultrasonic lens includes an inner surface defining a passage extending along a reference axis. The piezoelectric element is received in this passage and is acoustically coupled to the inner surface. The ultrasonic lens includes an outer surface opposite the inner surface, the outer surface including a shape with a concave profile. While positioned at the target location, the transducer device generates ultrasonic energy and ablates tissue at the target location by focusing the ultrasonic energy with the lens.

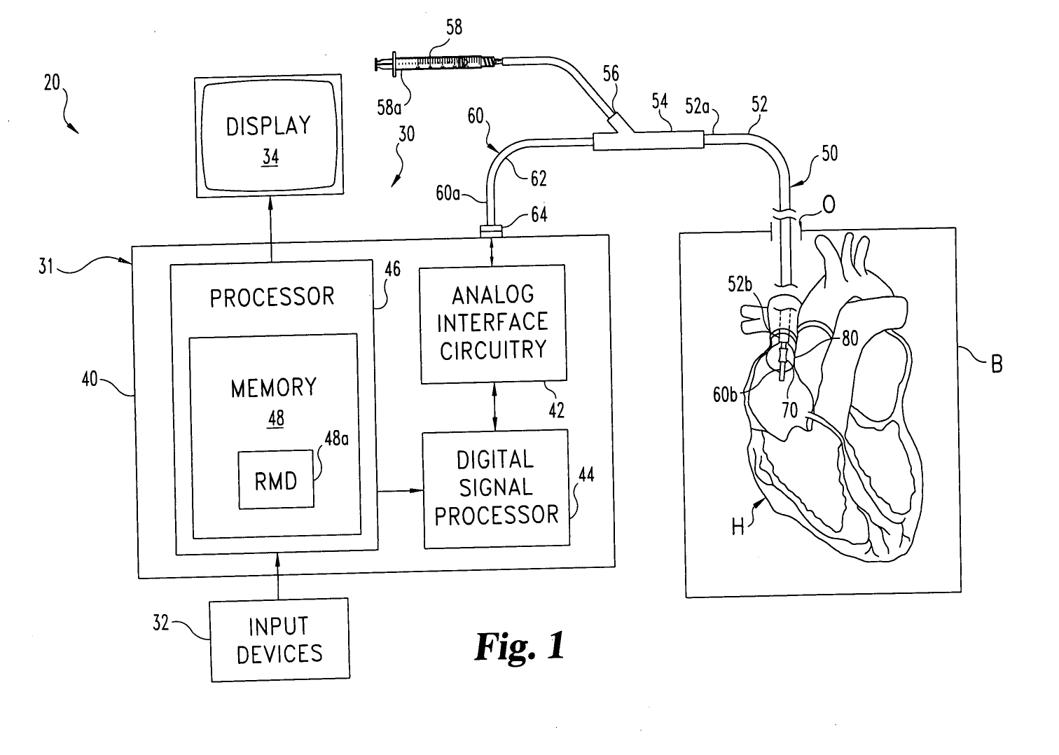

FIG. 1 illustrates system 20 that includes an endoscopically disposed ultrasonic transducer device and associated equipment arranged to provide medical treatment. System 20 is arranged to provide ultrasonic energy to body B, and more specifically to heart H, for medical treatment. System 20 includes control station 30, catheterization equipment 50, and ultrasonic probe 60. Control station 30 includes equipment 31 coupled to probe 60. Probe 60 is configured with catheterization equipment 50 for placement within body B of a human patient or subject, as schematically represented in FIG. 1. Equipment 31 includes operator input devices 32, operator display device 34, and various other operator-utilized equipment of system 20 that is external to body B of a patient during use. Input devices 32 include an alphanumeric keyboard and mouse or other pointing device of a standard variety. Alternatively or additionally, one or more other input devices can be utilized, such as a voice input subsystem or a different type as would occur to those skilled in the art. Operator display device 34 can be of a Cathode Ray Tube (CRT) type, Liquid Crystal Display (LCD) type, plasma type, Organic Light Emitting Diode (OLED) type, or such different type as would occur to those skilled in the art. Alternatively or additionally, one or more other operator output devices can be utilized, such as a printer, one or more loudspeakers, headphones, or such different type as would occur to those skilled in the art. Station 30 also can include one or more communication interfaces suitable for connection to a computer network, such as a Local Area Network (LAN), Municipal Area Network (MAN), and/or Wide Area Network (WAN) like the internet; a medical diagnostic device; another therapeutic device; a medical imaging device; a Personal Digital Assistant (PDA) device; a digital still image or video camera; and/or audio device, to name only a few. Operator equipment 30 can be arranged to show other information under control of the operator.

Equipment 31 also includes processing subsystem 40 for processing signals and data associated with system 20. Subsystem 40 includes analog interface circuitry 42, Digital Signal Processor (DSP) 44, data processor 46, and memory 48. Analog interface circuitry 42 is responsive to control signals from DSP 44 to provide corresponding analog stimulus signals to Probe 60. At least one of analog circuitry 42 and DSP 44 includes one or more digital-to-analog converters (DAC) and one or more analog-to-digital converters (ADC) to facilitate operation of system 20 in the manner to be described in greater detail hereinafter. Processor 46 is coupled to DSP 44 to bidirectionally communicate therewith, selectively provide output to display device 34, and selectively respond to input from operator input devices 32.

DSP 44 and/or processor 46 can be of a programmable type; a dedicated, hardwired state machine; or a combination of these. DSP 44 and processor 46 perform in accordance with operating logic that can be defined by software programming instructions, firmware, dedicated hardware, a combination of these, or in a different manner as would occur to those skilled in the art. For a programmable form of DSP 44 or processor 46, at least a portion of this operating logic can be defined by instructions stored in memory 48. Programming of DSP 44 and/or processor 46 can be of a standard, static type; an adaptive type provided by neural networking, expert-assisted learning, fuzzy logic, or the like; or a combination of these.

Memory 48 is illustrated in association with processor 46; however, memory 48 can be separate from or at least partially included in one or more of DSP 44 and processor 46. Memory 48 includes at least one Removable Memory Device (RMD) 48a. Memory 48 can be of a solid-state variety, electromagnetic variety, optical variety, or a combination of these forms. Furthermore, memory 48 and can be volatile, nonvolatile, or a mixture of these types. Memory 48 can be at least partially integrated with circuitry 42, DSP 44, and/or processor 46. RMD 48a can be a floppy disc, cartridge, or tape form of removable electromagnetic recording media; an optical disc, such as a CD or DVD type; an electrically reprogrammable solid-state type of nonvolatile memory, and/or such different variety as would occur to those skilled in the art. In still other embodiments, RMD 48a is absent.

Circuitry 42, DSP 44, and processor 46 can be comprised of one or more components of any type suitable to operate as described herein. Further, it should be appreciated that all or any portion of circuitry 42, DSP 44, and processor 46 can be integrated together in a common device, and/or provided as multiple processing units. For a multiple processing unit form of DSP 44 or processor 46; distributed, pipelined, and/or parallel processing can be utilized as appropriate. In one embodiment, circuitry 42 is provided as one or more components coupled to a dedicated integrated circuit form of DSP 44; processor 46 is provided in the form of one or more general purpose central processing units that interface with DSP 44 over a standard bus connection; and memory 48 includes dedicated memory circuitry integrated within DSP 44 and processor 46, and one or more external memory components including a removable disk form of RMD 48a. Circuitry 42, DSP 44, and/or processor 46 can include one or more signal filters, limiters, oscillators, format converters (such as DACs or ADCs), power supplies, or other signal operators or conditioners as appropriate to operate system 20 in the manner to be described in greater detail hereinafter.

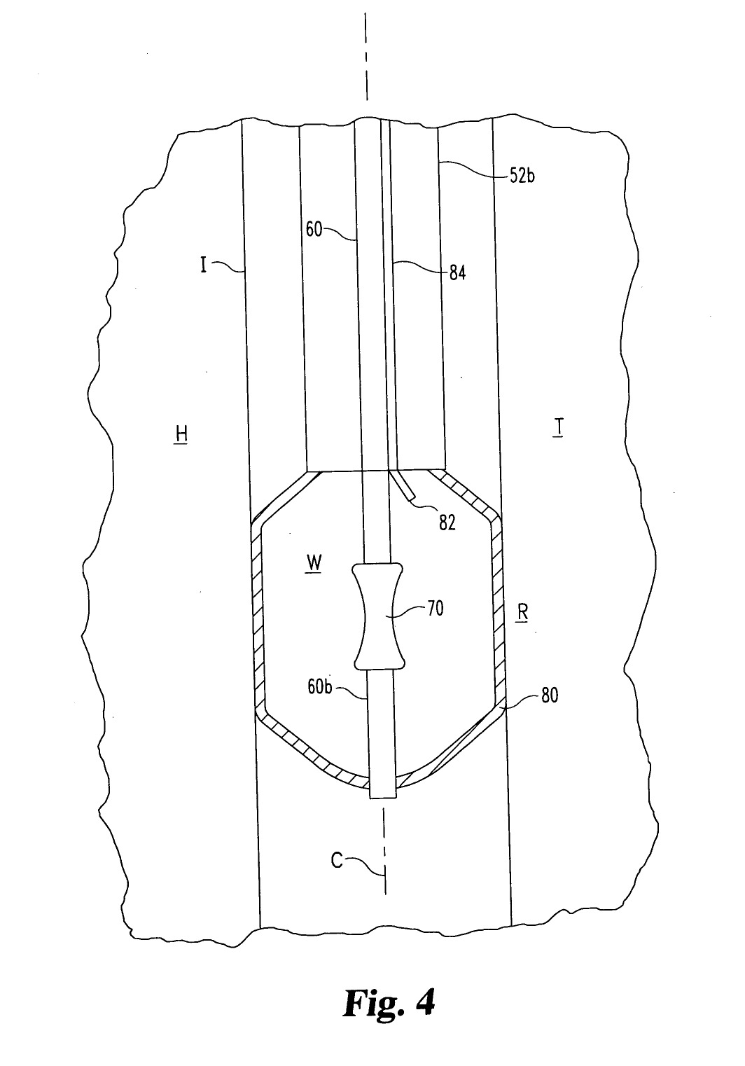

Referring also to FIG. 4, equipment 50 includes flexible catheter 52 with proximal end 52a opposite distal end 52b, and catheter port device 54. Catheter port device 54 can also be used as an operator handle when necessary. Proximal end 52a is connected to catheter port device 54 to be in fluid communication therewith. Catheter 52 includes one or more lumens extending therethrough. Equipment 50 is introduced into and removed from body B through opening O in a standard manner that typically includes one or more other components not shown to enhance clarity.

Probe 60 has proximal end portion 60a and distal end portion 60b. Probe 60 includes electrical cabling 62 with connector 64 electrically connected to equipment 31 of station 30. Cabling 62 extends from connector 64 at proximal end portion 60a through port device 54 and a lumen of catheter 52 to distal end portion 60b. Probe 60 carries transducer device 70 and terminates at the distal tip of distal end portion 60b. Transducer device 70 is connected to cabling 62 at distal end portion 60b. Additionally or alternatively to probe 60, a stint, other surgical instrument, or other type of cabling system can be utilized in the operation of system 20.

At proximal end portion 52a of catheter 52, balloon control port 56 is coupled to balloon control device 58. Distal end portion 60b of probe 60 includes balloon 80 that can be selectively expanded to maintain position once the target internal body area of the heart H is reached. Balloon 80 surrounds and encloses transducer device 70 carried at the distal end portion 60b, further aspects of which are described below.

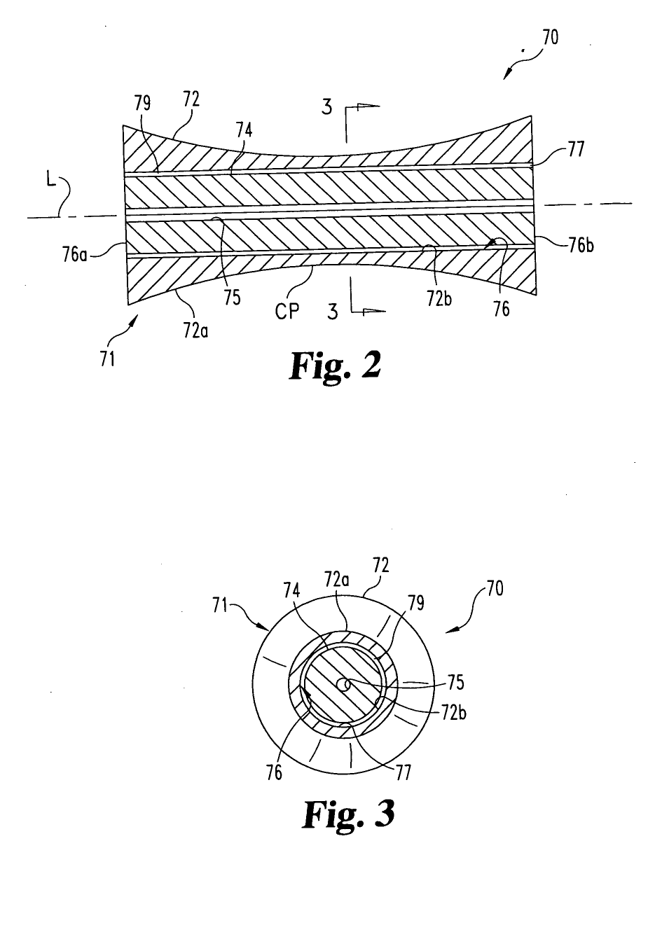

Additionally, FIGS. 2 and 3 illustrate transducer device 70 along centerline, longitudinal axis L. Transducer device 70 includes ultrasonic lens 72 and piezoelectric element 74. Element 74 of device 70 is coupled to cabling 62 and responds to electrical signals provided by cabling 62 to controllably generate ultrasonic energy. Ultrasonic lens 72 and piezoelectric element 74 are generally cylindrically shaped and symmetric about longitudinal axis L. Ultrasonic lens 72 has outer surface 72a opposite inner surface 72b. Outer surface 72a defines a concave profile CP of lens 72 to focus ultrasonic energy for tissue ablation. Outer surface 72a of lens 72 preferably includes a protective film covering, such as a paralyne coating (not shown). Lens 72 extends at least partially around longitudinal axis L. In the embodiment illustrated in FIGS. 2 and 3, lens 72 extends completely (360°) around axis L, forming a hyperboloid of one-sheet. Lens 72 can be composed of any appropriate material as would generally occur to one skilled in the art, such as a metal, a plastic, a composite, or a liquid or gas encapsulated to form a lens. In one embodiment, lens 72 is composed of aluminum or magnesium, wherein the aluminum or magnesium functions as an acoustic matching layer. Additionally, lens 72 may be a Fresnel lens to reduce the diameter of the device.

FIG. 3 illustrates a cross-sectional view of transducer device 70 along section line 3-3 shown in FIG. 2. As illustrated, piezoelectric element 74 is positioned interior to ultrasonic lens 72. Piezoelectric element 74 defines central channel 75 therethrough. Inner surface 72b of lens 72 defines passage 76 forming cavity 77 that is approximately shaped as a right circular cylinder. Cavity 77 is symmetric about axis L, which is shown by crosshairs in FIG. 3—being perpendicular to the view plane. Passage 76 (cavity 77) is open at each end of lens 72 forming respective apertures 76a, 76b. As illustrated in FIGS. 2 and 3, piezoelectric element 74 is received in passage 76 through one of apertures 76a or 76b to at least partially occupy cavity 77. This occupancy is partial, leaving gap 79. Lens 72 and element 74 are assembled together to maintain element 74 within cavity 77. As illustrated, ablation assembly 71 includes lens 72 and element 74 assembled together. In a preferred embodiment, the maximum cross-sectional diameter of transducer device 70 taken perpendicular to axis L is 20 millimeters (mm) or less. In a more preferred embodiment, this maximum cross-sectional diameter of transducer device 70 is 3 mm or less.

Ultrasonic lens 72 is acoustically coupled to piezoelectric element 74 by any appropriate method as would generally occur to one skilled in the art. In one form, a liquid is used to acoustically couple element 74 to lens 72 in gap 79. However, in other embodiments, one or more gases or solids could be used to provide the desired coupling. Additionally, transducer device 70 is operably and structurally connected to probe 60 by any appropriate method as would generally occur to one skilled in the art. In an alternative embodiment, system 20 includes multiple piezoelectric elements cooperating to operate as a transducer, such as device 170, that is more fully described hereinafter.

Referring to FIG. 4, a partial schematic, sectional view of distal end portion 60b is shown. As can best be seen in FIG. 4, distal end 52b of catheter 52 extends along longitudinal center axis C and includes transducer 70 and balloon fluid conduit 84. Conduit 84 is in fluid communication with balloon fluid port 82, end portion 606 and control port 56 at end portion 60a (not shown). Distal end portion 60b includes balloon 80, which is in fluid communication with balloon fluid port 82. The balloon 80 expands when a fluid W, such as a liquid or a gas, is introduced into the balloon 80 through the balloon fluid port 82. The balloon 80 expands to a point where it is in contact with an interior wall I of heart H to direct ultrasonic energy to tissue T in region R. This expansion maintains transducer 70 in a generally fixed orientation relative to tissue T in region R. Balloon 80 can be collapsed by removing the fluid through port 82 and conduit 84 to again move transducer 70 relative to tissue T.

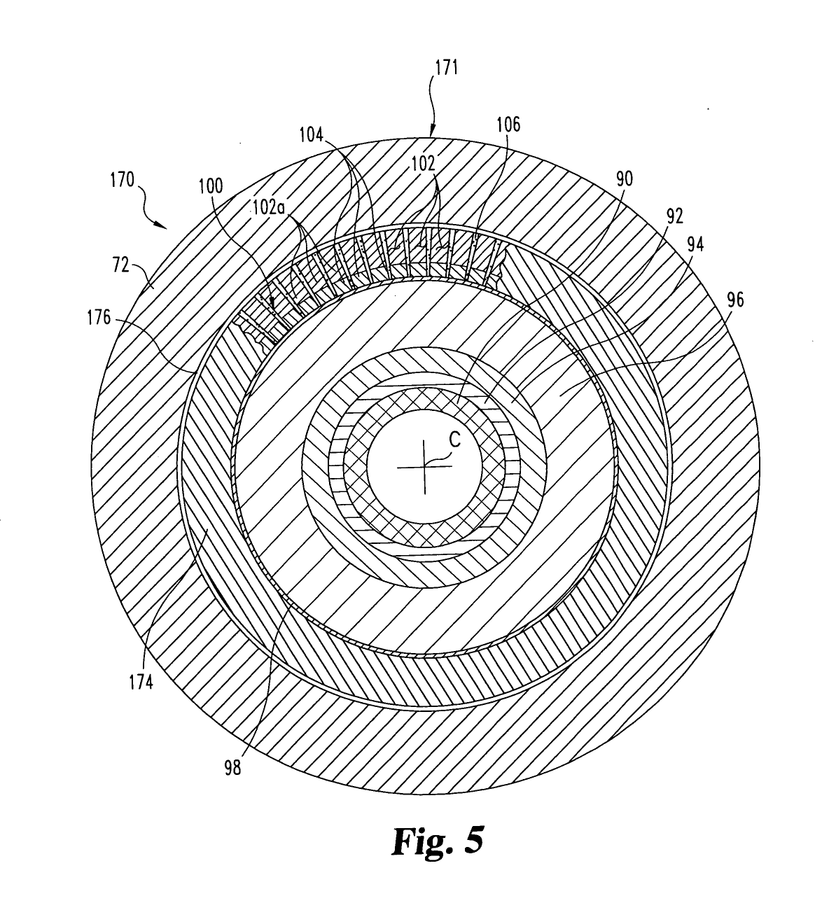

As an alternative to transducer 70, ablation transducer device 170 is shown in FIG. 5; where like reference numerals refer to like features. FIG. 5 presents a cross-sectional view of transducer device 170 taken perpendicular to its longitudinal center axis C. Transducer device 170 can be used as a substitute for transducer 70 shown in FIGS. 1-4 with adaptation made to operate with more than one piezoelectric element as further described hereinafter. Transducer device 170 includes ultrasonic lens 72 and flexible substrate 98 that carries piezoelectric element array 174. Flexible substrate 98 is preferably of a flexible circuit type. The various components of transducer device 170 are shaped generally in the form of a right circular cylinder in FIG. 5. A liquid gap 176 is positioned between ultrasonic lens 72 and piezoelectric element array 174. Alternatively, it should be appreciated that an acoustic matching layer of gas or solid material can be positioned between ultrasonic lens 72 and piezoelectric element array 174. An acoustic layer 96 is positioned substrate 98 and a second acoustic layer 94. Acoustic layer 94 is in contact with a cylindrical backing member 92. Member 92 surrounds a supporting core 90. It should be appreciated that acoustic layers 94 and 96, backing member 92, and supporting core 90 can be arranged differently in transducer device 170 as would generally occur to one skilled in the art. In another embodiment, one or more of the acoustic layers 94 and 96, backing member 92, and supporting core 90 are absent from transducer device 170.

Array 174 is formed by dividing one or more larger piezoelectric blocks into two or more elements 102 carried on the flexible substrate 98 of transducer device 170. Array 174 is shaped generally in the form of a right circular cylinder by wrapping substrate 98 about a like-shaped mandrel. Elements 102 each respond to an appropriate electrical stimulus to generate acoustic energy in the ultrasonic frequency range. Elements 102 are each generally rigid relative to flexible substrate 98 and are elongate with a longitude generally parallel to center axis C. Elements 102 are each generally sized and shaped the same, and are evenly spaced apart from one another. Transducer device 170 is alternatively designated ablation assembly 171.

In FIG. 5, center axis C is generally perpendicular to the view plane and is accordingly represented by cross-hairs that intersect at the origin of the circular cross section of transducer device 170. Correspondingly, center axis C is centrally located relative to array 174 in FIG. 5. Piezoelectric elements 102 are generally equidistant from center axis C, being spaced approximately evenly thereabout. In a preferred embodiment of the present application, elements 102 number 24 or more. In a more preferred embodiment, elements 102 number 64 or more. In an even more preferred embodiment, elements 102 number at least 256. Elements 102 can each be made of the same piezoelectric material. Alternatively, one or more elements 102 can be made of material different than one or more other of elements 102. Piezoelectric elements 102 are connected to metallic electrically conducting contacts 104 carried on substrate 98. In one form, connection between elements 102 and contacts 104 is made with an epoxy that does not unacceptably impede electrical contact. Contacts 104 are interior to elements 102 and are in contact with substrate 98. In this embodiment, transducer device 170 includes a support matrix material 106 between adjacent elements 102.

Substrate 98 preferably carries one or more electrically conductive traces. In the alternative embodiment incorporating ablation array 174, there are preferably a corresponding number of electrically conductive traces as to the number of elements 102. Additionally, cabling 62 carries a corresponding number of conductors which make electrical contact with the one or more electrically conductive traces. The electrical contact creates an electrical signal pathway to each of elements 102. In one embodiment, substrate 98 has two or more levels of electrically conductive traces, separated by electrical insulation. In another embodiment, a signal pad is operably connected to substrate 98 and makes electrical connection with a signal conductor disposed within cabling 62 to enable operation of transducer device 170.

In one embodiment, material 106 is a standard epoxy and acoustic layers 94 and 96 are formed from a thermoplastic and/or thermoset polymeric resin, such as parylene C polymer, selected to minimize transmission of ultrasonic energy from piezoelectric element 74 or array 100 towards core 90. In another embodiment, the same composition is used for both material 106 and acoustic layers 94 and 96. In still other embodiments, one or more other materials or backing structures and/or support matrix materials 106 are used as would occur to those skilled in the art. In other embodiments, acoustic layers 94 and 96 are formed from metals such as aluminum, silicon, or tungsten, for example; or are absent, with the corresponding space being filled by air.

Referring generally to FIGS. 1-4 and 6, one mode of operating system 20 is next described. During normal use, distal end portion 60b of probe 60 is inserted through opening O of body B, and advanced through passageway P to heart H. The insertion and advancement of probe 60 through body B is performed with a standard catheterization procedure using catheter 52. Typically, passageway P is defined by the circulatory system vasculature of body B. Probe 60 is navigated through passageway P to target tissue region R in heart H. Region R includes cardiac tissue. A guide wire can be utilized in through channel 75 to navigate device 70 to region R. A guide wire is typically navigated to the target location in advance of catheter 52. Catheter 52 is then advanced with device 70. Alternatively, device 70 is subsequently advanced through catheter 52. In still other alternatives, device 70 is slidably moved along a previously placed guide wire without utilization of catheter 52, and/or device 70 is of a self-directing, steerable variety that does not require a catheter or guide wire to navigate body passageways to a target site within the patient.

Thereafter, balloon control device 58, which is in the form of a syringe 58a and is coupled to balloon fluid port 82, is operated to distribute liquid, such as fluid W, under pressure through balloon control port 56 into balloon fluid conduit 84. Fluid W from conduit 84 enters balloon 80 through fluid port 82 and expands balloon 80 to hold balloon 80 in a selected position along interior wall I of heart H to generally fix transducer device 70 in passageway P relative to region R, with transducer device 70 being generally centered in passageway P.

After positioning, piezoelectric element 74 of transducer device 70 is controllably activated with operator equipment 30 to selectively ablate tissue T of interior wall I of heart H by application of acoustic power from piezoelectric element 74 in the ultrasonic range through balloon 80 and fluid W inside balloon 80. In a preferred embodiment, the ultrasonic energy has a frequency in the range of 1 MegaHertz (MHz) to 20 MHz. In an alternative embodiment, array 174 of transducer device 170 is controllably activated with operator equipment 30 to selectively ablate tissue T on interior wall I of heart H by application of acoustic power from one or more of elements 102 in the ultrasonic range through balloon 80 and fluid W inside balloon 80, as will be discussed below in greater detail. For either device 70 or 170, lens 72 focuses the ultrasonic energy to ablate tissue in a narrowly focused area along at least a portion of a circumference about transducer device 70. Preferably, a circumferential ring of ablated tissue about transducer device 70 or 170, respectively, results.

It should be appreciated that other components, devices, and systems can be integrated into system 20, such as an endoscope system, an imaging system, a lighting system, and/or a video camera system, to name a few examples. In one alternative embodiment, an endoscope (not shown) is integrated into system 20. Distal end portion 60b is navigated through opening O into heart H to the desired internal wall I utilizing images conveyed through a port to operator equipment 30 via an image communication pathway. These images may be displayed with display device 34. Light to facilitate visualization in this way may be provided from a light source that is coupled to a port via a light pathway.

In another alternative embodiment, system 20 can be operated in a mode to determine the location of transducer device 70 relative to a region in the body B to verify proper positioning. In one mode of operation, transducer device 70 generates an ultrasonic signal of 20 Mhz or less that is reflected back to and detected by transducer device 70. The reflected signal is processed by subsystem 40 to determine the distance from transducer device 70 to the interface of balloon 80 and tissue T. This locating information is used to direct high intensity focused ultrasound (HIFU) energy to the desired target area. In one particular mode, this operating mode can be used to generate an ultrasonic image to assist with positioning. This relative position determination can be performed before, during, and after balloon expansion, as desired. Further, this mode can be executed before and after a tissue ablation mode of operation of transducer device 70.

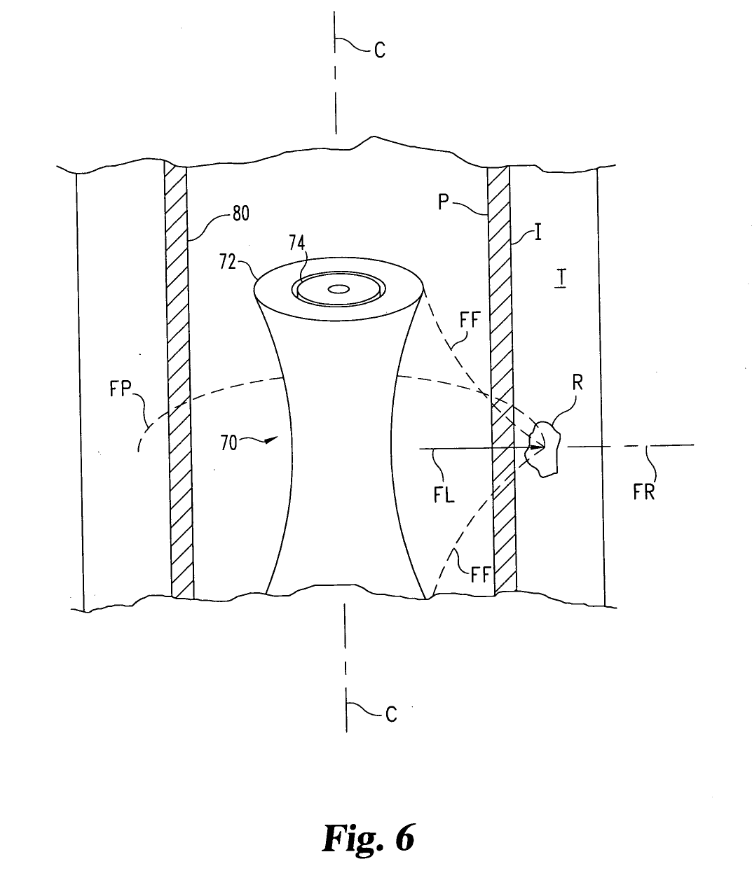

Referring to FIG. 6, ultrasonic lens 72 and piezoelectric element 74 of transducer device 70 are schematically shown to illustrate the spatial relationship of lens 72 and element 74 with respect to tissue T. The various other components of transducer device 70 are not illustrated in FIG. 6 to preserve clarity. Due to the concave outer profile of ultrasonic lens 72 revolved about center axis C, a relatively narrowly focused region of ultrasonic acoustic power can be concentrated on region R of heart H. In FIG. 6, the focused ultrasonic energy is represented by focal lines FF with focal length FL along focal axis FR represented by a radial ray. In a preferred embodiment, the focal length FL is in the range of 1 millimeter (mm) to 60 mm. It should be appreciated that the focal point is located behind the surface of interior wall I. Ultrasonic lens 72 focuses the ultrasonic energy along focal perimeter FP shown in FIG. 6 to form a ring-shaped ablation legion in the circumferentially surrounding tissue T of interior wall I.

In an alternative mode of ablation operation involving ablation array 174 in place of element 74, different subsets of elements 102 are activated in a selected sequence in accordance with operating logic of subsystem 40. In one preferred embodiment, sixty-four (64) consecutive elements 102 are activated at one time corresponding to a 90 degree or less angular aperture. By controlling relative phase and magnitude of an oscillatory electrical stimulus (such as a sinusoidal waveform) to each of the activated elements, a relatively narrowly focused region of ultrasonic acoustic power can be concentrated on region R of heart H. In one implementation, different subsets of elements 102 are sequentially activated to focus the ultrasonic energy along focal perimeter FP shown in FIG. 6. This sweep can continue for 360° to form a ring-shaped ablation legion in the circumferentially surrounding tissue T of interior wall I. Alternatively, the sweep can be less than 360° corresponding to a curved segment of ablated tissue based on activation of less than all of the elements 102. Additionally or alternatively, the elements 102 can be activated to form ablated segments spaced apart from one another along perimeter FP.

After ablation at a fixed location has been accomplished, fluid W is withdrawn from balloon 80 with control device 58 via port 56, port 82, and conduit 84. By removing fluid W, balloon 80 collapses and can be moved to a different location to perform ablation again, or can be withdrawn from body B of the patient. Indeed, in one application, it is envisioned that ablation will occur at several different locations to reduce or eliminate undesirable electrical signals being sent through cardiac tissue. Such applications include arterial fibrillation, for which the application may alternatively or additionally extend to ablation of regions in a pulmonary vein or the like. Nonetheless, in other applications and/or embodiments, system 20 may be used in a different manner and/or in a different location internal to body B. After all internal applications are complete, probe 60 is withdrawn from the body B of the patient.

Many other embodiments of the present invention are envisioned. Indeed, different ways of shaping, filling, and the like can be used. In still other embodiments a different kind of noncylindrical shape of piezoelectric element 74 and/or array 174 can be provided in lieu of the generally flat, planar form illustrated. Alternatively or additionally, other materials, shapes, sizes, and designs can be utilized in connection with a flexible circuit substrate comprised of one or more layers with direct coupling to electrical signal pads via cabling.

All publications, patents, and patent applications cited in this specification are herein incorporated by reference as if each individual publication, patent, or patent application were specifically and individually indicated to be incorporated by reference and set forth in its entirety herein. Any theory, mechanism of operation, proof, or finding stated herein is meant to further enhance understanding of the present invention and is not intended to make the present invention in any way dependent upon such theory, mechanism of operation, proof, or finding. While the invention has been illustrated and described in detail in the drawings and foregoing description, the same is to be considered as illustrative and not restrictive in character, it being understood that only the selected embodiments have been shown and described and that all changes, modifications, and equivalents of the inventions as defined herein or by the following claims are desired to be protected.

1. A method, comprising:
advancing a device through a passageway inside a patient's body toward a target location, the device including a piezoelectric element and an ultrasonic lens, the ultrasonic lens including an inner surface defining a passage, the piezoelectric element being received in the passage and being acoustically coupled to the inner surface, the ultrasonic lens including an outer surface opposite the inner surface, the outer surface defining a shape with a concave profile;
after the advancing, maintaining the device in a selected position in the passageway to orient the concave profile relative to the target location;
while the concave profile is oriented relative to the target location, generating ultrasonic energy with the piezoelectric element of the device; and
ablating tissue at the target location by focusing the ultrasonic energy with the concave profile of the lens.

2. The method of claim 1, wherein the passage extends along a reference axis and the concave profile is revolved at least partially about the reference axis.

3. The method of claim 2, wherein the ultrasonic lens extends completely (360 degrees) about the reference axis and the ablating includes ablating tissue corresponding to a ring shape positioned about the ultrasonic lens.

4. The method of claim 2, wherein the transducer device is arranged in a cylindrical shape about the reference axis.

5. The method of claim 1, further comprising a plurality of piezoelectric elements positioned inside the passage of the lens.

6. The method of claim 1, wherein the piezoelectric element is at least partially composed of a piezoceramic material.

7. The method of claim 1, wherein the device further includes fluid contained in the passage of the ultrasonic lens between the piezoelectric element and the inner surface of the ultrasonic lens.

8. The method of claim 1, wherein the maintaining includes expanding a balloon to make contact with the passageway, the device being positioned inside the balloon.

9. The method of claim 8, which includes collapsing the balloon after the ablating is completed to withdraw the device from the patient's body.

10. The method of claim 1, wherein the ultrasonic lens is comprised of at least one of aluminum and magnesium.

11. The method of claim 1, wherein a maximum cross sectional dimension of the transducer device taken perpendicular to the reference axis is 20 millimeters.

12. The method of claim 1, wherein the focused ultrasonic energy has a focal length in a range of 1 to 60 millimeters.

13. The method of claim 1, wherein the passageway extends through the heart of the patient's body and the target location includes cardiac tissue.

14. The method of claim 13, which includes repositioning the device to ablate a portion of the cardiac tissue at a different target location.

15. A method, comprising:
providing a piezoelectric element that is approximately symmetric about a centerline axis longitudinally extending along the piezoelectric element;
providing an ultrasonic lens that includes an inner surface defining a passage and an outer surface defining a shape with a concave profile, the ultrasonic lens being approximately symmetric about a reference axis extending through the passage;
placing the piezoelectric element in the passage to acoustically couple with the inner surface of the ultrasonic lens to provide an ablation assembly; and
structuring the piezoelectric element and the ultrasonic lens to focus ultrasonic energy generated with the piezoelectric element in accordance with the concave profile to ablate material corresponding to a ring about the ablation assembly.

16. The method of claim 15, further comprising positioning the assembly inside a balloon that is expandable to maintain a position of the ultrasound device.

17. The method of claim 15, wherein the piezoelectric element is generally shaped as a right circular cylinder.

18. The method of claim 15, further comprising providing a probe including a proximal end portion opposite a distal end portion, the ablation assembly being carried at the distal end portion, the probe being structured for advancement and withdrawal through a passageway inside a patient's body.

19. The method of claim 18, wherein cabling is carried inside the probe.

20. The method of claim 15, wherein the ultrasonic lens is comprised of at least one of aluminum and magnesium.

21. The method of claim 15, wherein the focused ultrasonic energy has a focal length in a range of 1 to 60 millimeters.

22. The method of claim 15, wherein the ultrasonic lens is a Fresnel type lens.

23. The method of claim 15, wherein the ultrasonic lens is shaped as a hyperboloid of one sheet.

24. The method of claim 15, wherein the ablation assembly is structured to acoustically couple the piezoelectric element to the inner surface of the lens with a fluid positioned therebetween.

25. A system, comprising:
a probe with a distal end portion opposite a proximal end portion, the probe including cabling and being structured to advance through a passageway of a patient's body to a target location including cardiac tissue;
an ablation assembly included with the probe at the distal end portion to be carried therewith to the target location, the assembly including a piezoelectric element coupled to the cabling and an ultrasonic lens, the ultrasonic lens including an inner surface defining a cavity and an outer surface shaped with a concave profile, the piezoelectric element being positioned in the cavity and acoustically coupled to the inner surface of the lens;
a controller to selectively activate and deactivate the piezoelectric element, the controller being coupled to the cabling at the proximal end portion of the probe and being structured for placement external to the patient's body while the ablation assembly is positioned at the target location; and
wherein the assembly is responsive to the controller to selectively generate ultrasonic energy with the piezoelectric element and is structured to focus the ultrasonic energy at a focal length determined in accordance with the concave profile and ablate the cardiac tissue with the ultrasonic energy when the piezoelectric element is activated and the ablation assembly is positioned at the target location.

26. The apparatus of claim 25, wherein the ablation assembly has a maximum cross sectional dimension of 4 millimeters or less taken through the piezoelectric element perpendicular to longitude of the probe.

27. The apparatus of claim 25, wherein the ultrasonic lens has a hyperbolic shape.

28. The apparatus of claim 25, wherein the ultrasonic lens is a Fresnel type lens.

29. The apparatus of claim 25, further comprising a balloon, the piezoelectric element being positioned inside the balloon, the balloon being structured to selectively expand to maintain the ablation assembly at the target location and collapse to selectively move the ablation assembly.

30. The apparatus of claim 25, wherein the lens has a shape including the concave profile revolved about a reference axis extending through the cavity.

31. The apparatus of claim 25, wherein the ultrasonic lens is configured as a hyperboloid of one-sheet.

32. The apparatus of claim 25, wherein the ablation assembly further includes fluid between the piezoelectric element and the inner surface of the ultrasonic lens.
